(19) United States
(12) Patent Application Publication
(10) Pub. No.: US 2024/0125691 A1
(43) Pub. Date: Apr. 18, 2024

(54) METHOD OF MEASURING BIOLOGICAL INFORMATION, AND MEASURING SYSTEM

(71) Applicants: Yoshihiro OBA, Miyagi (JP); Ryosuke KASAHARA, Kanagawa (JP); Toshihide SASAKI, Miyagi (JP); Yoshio WADA, Kanagawa (JP)

(72) Inventors: Yoshihiro OBA, Miyagi (JP); Ryosuke KASAHARA, Kanagawa (JP); Toshihide SASAKI, Miyagi (JP); Yoshio WADA, Kanagawa (JP)

(21) Appl. No.: 18/277,803

(22) PCT Filed: Mar. 4, 2022
(86) PCT No.: PCT/JP2022/009559

§ 371 Date: Aug. 18, 2023

(30) Foreign Application Priority Data
Mar. 8, 2021 (JP) 2021-036544

(51) Int. Cl. G01N 21/15; G01N 21/552

(52) U.S. Cl. CPC G01N 21/15; G01N 21/552; G01N 2021/151; G01N 2021/152; G01N 2021/157; G01N 2201/12707

(57) ABSTRACT

A biological information measuring technique with improved reliability is proposed. A method of measuring biological information using an optical element with a totally reflecting surface, which is to be brought into contact with an object to be observed, is provided. The method includes, after the optical element has been in contact with the object to be observed, acquiring an absorbance at the totally reflecting surface in a state where the optical element is not in contact with the object to be observed, and determining by an information processing apparatus whether the object to be observed needs to be cleaned, based on the absorbance.

### TECHNICAL FIELD

The present invention relates to a method of measuring biological information and a measuring system.

### BACKGROUND ART

In recent years, the number of diabetic patients is increasing all over the world, and non-invasive blood glucose monitoring, without blood sampling, is desired. For optical sensing of glucose, various schemes have been proposed, including monitoring with near-infrared spectroscopy, mid-infrared spectroscopy, Raman spectroscopy, etc. Among these, the mid-infrared region is the fingerprint region in which glucose absorption is significant and the measurement sensitivity is high compared with the near-infrared region.

It is proposed, in monitoring a glucose level or concentration using an attenuated total reflection (ATR) prism, to monitor the spectra at wavenumbers 1035 cm⁻¹, 1080 cm⁻¹, and 1110 cm⁻¹, corresponding to the absorption peaks of glucose. See, for example, Japanese Patent No. 5,376,439. A technique for reducing the frequency of acquiring background spectrum during Fourier Transform Infrared (FTIR) spectroscopy or FTIR-ATR spectroscopy is also known. See, for example, Japanese Paten No. 6,683,682.

### SUMMARY OF INVENTION

### Technical Problem

With the conventional techniques for measuring biological information, there is room for improving the measuring accuracy under the situation where the same ATR prism is repeatedly used. It is one of the objectives to provide a biological information measuring technique with improved measuring accuracy.

### Solution to Problem

In one aspect, a method of measuring biological information using an optical element with a totally reflecting surface, which is to be brought into contact with an object to be observed, is provided. The method includes,
after the optical element has been in contact with the object to be observed, acquiring an absorbance at the totally reflecting surface in a state where the optical element is not in contact with the object to be observed, and
determining by an information processing apparatus whether the object to be observed needs to be cleaned, based on the absorbance.

In another aspect, a method of measuring biological information using an optical element with a totally reflecting surface, which is to be brought into contact with an object to be observed, includes
before the optical element comes into contact with the object to be observed, acquiring an absorbance at the totally reflecting surface at a first wavenumber, and
determining by an information processing apparatus whether or not the optical element needs to be cleaned based on the absorbance at the first wavenumber, the first wavenumber being in a range of 1700 cm⁻¹ to 1800 cm⁻¹, or a range of 2800 cm⁻¹ to 3000 cm⁻¹.

### Advantageous Effects of Invention

The above-described schemes can improve the reliability of biological information measurement. Other objects and further features of the present disclosure will be apparent from the following detailed description when read in conjunction with the accompanying drawings.

### BRIEF DESCRIPTION OF DRAWINGS

FIG. 1 is a schematic diagram of a measuring system according to an embodiment;

FIG. 2 is a block diagram of an information processing apparatus used in the system of FIG. 1;

FIG. 3 is a flowchart of a measuring process according to an embodiment;

FIG. 4A shows how to determine the necessity of cleaning the ATR prism;

FIG. 4B shows how to determine the necessity of cleaning the ATR prism;

FIG. 5A shows how to determine the necessity of cleaning the object to be observed;

FIG. 5B shows how to determine the necessity of cleaning the object to be observed;

FIG. 6 is an enlarged view of the wavenumber range from 1000 cm⁻¹ to 2000 cm⁻¹ shown in FIG. 5B;

FIG. 7 is an enlarged view of the wavenumber range from 2000 cm⁻¹ to 3000 cm⁻¹ shown in FIG. 5B;

FIG. 8 shows an absorption spectrum of vegetable oil;

FIG. 9 is a flowchart of a cleaning process according to an embodiment;

FIG. 10A shows a cleaning process using cleaning equipment according to an embodiment;

FIG. 10B shows the cleaning process using the cleaning equipment according to the embodiment;

FIG. 10C shows the cleaning process using the cleaning equipment according to the embodiment;

FIG. 11 shows a cleaning strategy for cleaning an ATR prism;

FIG. 12 shows another cleaning strategy for cleaning an ATR prism;

FIG. 13A shows a modification of the ATR prism;

FIG. 13B shows another modification of the ATR prism; and

FIG. 13C shows still another modification of the ATR prism.

### DESCRIPTION OF EMBODIMENTS

In the embodiment, automatic determination is made as to whether an object to be observed needs to be cleaned for biological information measurement. In another aspect, automatic determination is made as to whether an optical element used for the biological information measurement needs to be cleaned. If it is determined that the object to be observed needs to be cleaned, a signal for encouraging cleaning of the object to be observed is output. If it is determined that the optical element needs to be cleaned, cleaning equipment is automatically driven to clean up the optical element. By performing the above-described determination as to the necessity of cleaning, measurement accuracy and reliability can be improved. In the following description, the same or corresponding elements are designated by the same reference numerals, and explanations of the same or similar elements will not be provided.

### System Configuration

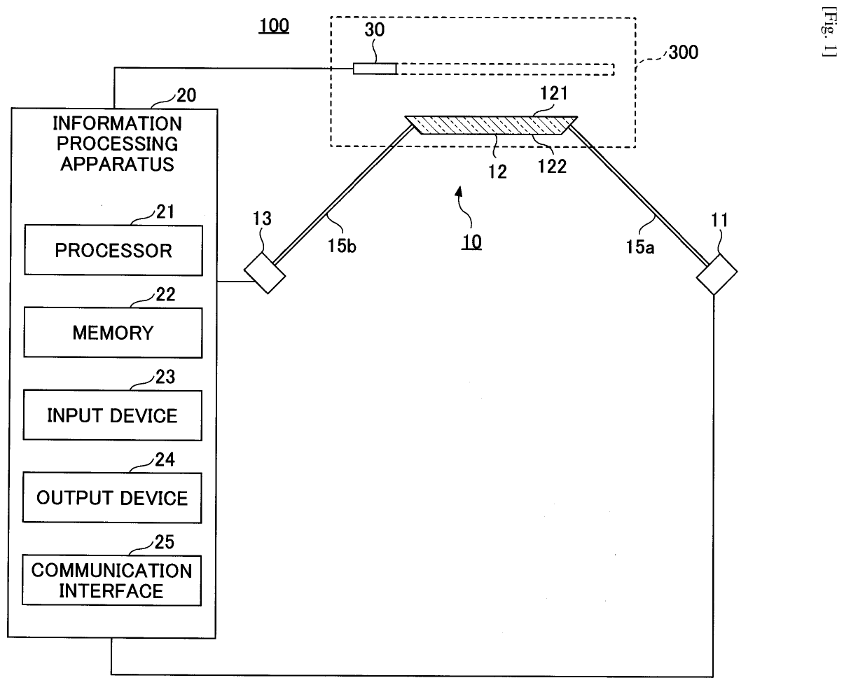

FIG. 1 is a schematic diagram of a measuring system 100 according to an embodiment. The measuring system 100 includes a measuring device 10, an information processing apparatus 20, and cleaning equipment 30. The measuring device 10 includes an ATR prism 12, a multi-wavelength light source 11, and a photodetector 13. The multi-wavelength light source 11 is connected to the light incident side of the ATR prism 12 through an optical fiber 15a. The photodetector 13 is connected to the light emission side of the ATR prism 12 through an optical fiber 15b. The ATR prism 12 has totally reflecting surfaces 121 and 122.

The multi-wavelength light source 11 emits infrared light at multiple wavelengths. The term “wavelength” is synonymous with “wavenumber” in the following description, because wavelength is the reciprocal of the wavenumber. The multiwavelength light source 11 may be configured with a plurality of light sources with different wavelengths, such that a desired wavelength is selected by means of a switch or the like. Alternatively, a light source with a broad wavelength range, such as a lamp light source, a light emitting diode (LED), a super luminescent diode (SLD), etc., may be used. If the latter case, a wavelength filter may be provided at the emission side of the light source to extract a desired wavelength, as necessary, or a plurality of light receiving elements may be provided in the photodetector 13 in combination with a wavelength filter provided at the incident side of each of the light receiving elements.

The ATR prism 12 is made of a high refractive index material, and the totally reflecting surfaces 121 and 122 are provided at the interface between the ATR prism 12 and the external medium. The light incident onto the input facet of the ATR prism 12 from the optical fiber 15a travels inside the prism, while being totally internally reflected at the totally reflecting surfaces 121 and 122. ATR measurement is a measuring scheme that makes use of the evanescent field “leaking” into the adjacent environment, which occurs when light is totally internally reflected at the interface between the prism and the object to be observed. When the ATR prism 12 is pressed against the object being observed for the biological measurement, the evanescent field leaking from the ATR prism 12 is absorbed by the object.

In the measurement of the intensity of a light absorption spectrum making use of the evanescent field, the ATR prism 12 is often held between upper and lower lips. This is because the stratum corneum of a mucous membrane, such as a lip surface, is very thin, and because a biological substance such as glucose contained in body fluid or blood can be detected with sufficient sensitivity. However, residues from hoods, lipsticks, lip balms, or saliva tend to be left on the lip surface. If such residue adheres to the ATR prism 12, it is difficult to identify the target signal derived from the biological substance in the living body. The same thing happens when a very thin skin surface, such as an eyelid, an earlobe, or a fingertip is monitored. In this case, sebum, sweat, cosmetics ingredients, or the like may affect the ATR measurement. In order to maintain the measuring reliability, it is necessary to keep the ATR prism 12 clean before and after the measurement.

It may be conceived that the ATR prism 12 is replaced every time measurement is made. However, in ATR measurements, the depth of penetration of the evanescent field into the observed object is defined according to the placement or the setup position of the ATR prism 12. If the ATR prism 12 is replaced, there is a strict requirement for reproducibility of the prism setup position, and the consistency of the absorption distribution on the prism surface has to be reevaluated and confirmed every time the prism is replaced. Meanwhile, if a user manually cleans the ATR prism 12 at his/her own discretion, the cleanup level varies, and accordingly, the measurement accuracy varies.

In the embodiment, the information processing apparatus 20 determines whether either the ATR prism 12 or the object to be observed needs to be cleaned. If cleaning of the ATR prism 12 is required, the cleaning equipment 30 automatically purifies the ATR prism 12. If the object to be observed needs to be cleaned, the information processing apparatus 20 outputs a signal or a message to encourage the user to clean up the observed part.

The information processing apparatus 20 includes a processor 21, a memory 22, an input device 23, an output device 24, and a communication interface 25, at least. The output of the photodetector 13 is connected to the input of the processor 21 so that an absorption spectrum is generated. The memory 22 stores a background spectrum as the reference spectrum, which may be acquired in advance. The memory 22 may record the measurement result detected by the measuring device 10. The memory 22 also stores parameters, programs, or the like required for the operation of the information processing apparatus 20.

The input device 23 may be a touch panel, a keyboard, a mouse, or the like, to input a user's manipulation. The output device 24 includes a display device, a speaker, and other output tools so as to display a message, or output a voice message for encouraging the user to clean up the observed part. The communication interface 25 communicates with the multi-wavelength light source 11 and with the cleaning equipment 30, and transmits and receives control signals, operating state information, etc.

The communications between the information processing apparatus 20 and the multi-wavelength light source 11, and between the information processing apparatus 20 and the cleaning equipment 30 may either be wired communication using a cable or a wireless one. The information processing apparatus 20 may be a desktop personal computer, or a mobile terminal such as a smartphone or a cellular tablet.

The cleaning equipment 30 is provided in, for example, a clean work station 300. When cleaning of the ATR prism 12 is required, a necessary cleanup process is carried out. At the clean work station 300, the ATR prism 12 is cleaned while it is still connected to the measuring device 10. Details of the cleaning equipment 30 and the cleanup process will be described later.

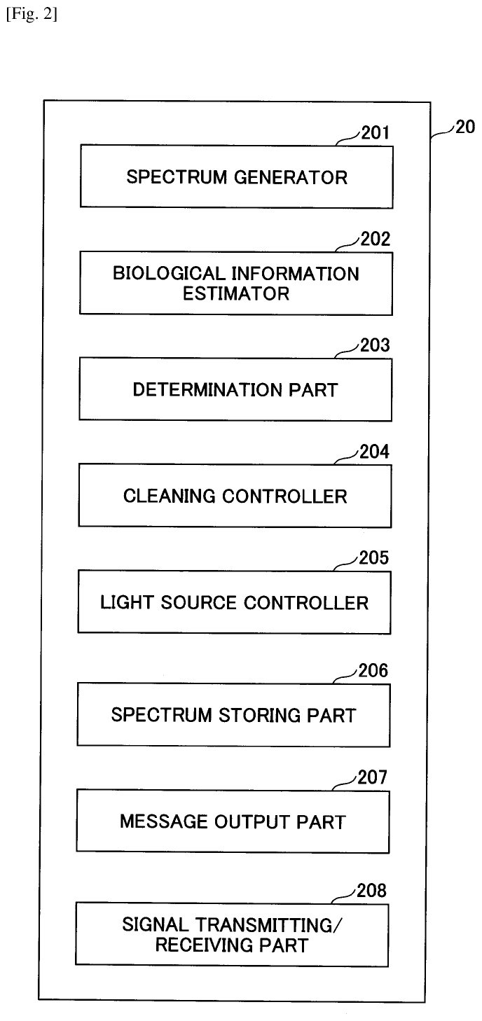

FIG. 2 is a functional block diagram of the information processing apparatus 20. The information processing apparatus 20 has, as functional blocks, a spectrum generator 201, a biological information estimator 202, a determination part 203, a cleaning controller 204, a light source controller 205, a spectrum storage part 206, a message output part 207, and a signal transmitting/receiving part 208.

The spectrum generator 201 calculates the absorbance of the observed object at each wavenumber based on the light intensity detected by the photodetector 13 at each wavenumber. Absorbance A at wavenumber k is expressed by the following formula

A(k)=−log₁₀(I/I₀) (1),

Where I₀ is the intensity of the incident light onto the ATR prism 12, that is, the intensity of the light emitted from the multi-wavelength light source 11, and I is the intensity of light detected by the photodetector 13. By determining the absorbance A over a predetermined range of wavenumbers, an absorption spectrum of the observed object can be obtained.

The biological information estimator 202 estimates the target biological information based on the absorption spectrum. The biological information is, for example, the glucose concentration in the blood (i.e., the blood glucose level). The determination part 203 determines whether or not the observed object or the ATR prism 12 needs to be cleaned, based on the absorbance or the absorption spectrum generated by the spectrum generator 201. If it is determined that the ATR prism 12 needs to be cleaned, the cleaning controller 204 drives and causes the cleaning equipment 30 to carry out the cleaning process for the ATR prism 12.

The light source controller 205 controls the on/off operation of the multi-wavelength light source 11, switching of the wavelength, etc. The light source controller 205 may control the acquisition timing of the detection result of the photodetector 13, synchronized with the wavelength changing timing of the light source. The operations of the spectrum generator 201, the biological information estimator 202, the determination part 203, the cleaning controller 204, and the light source controller 205 are implemented by the processor 21 of FIG. 1.

The spectrum storing part 206 stores a background spectrum as a reference, which has been acquired in advance. Absorption spectra acquired as the measurement results of the measurement device 10 may also be stored. The function of the spectrum storing part 206 is implemented by the memory 22 of FIG. 1.

The message output part 207 outputs a message or a signal encouraging cleanup of the observed object, when it is determined that the observed object needs to be cleaned. The message may be displayed on the display device or output as a voice message. Alternatively, a specific operation, such as lighting a particular lamp or sounding a chime, may be executed. Messages may be output not only when the cleanup of the observed object is required, but also when the ATR prism 12 needs to be cleaned. During the cleanup process of the ATR prism 12, a message such as “cleaning in progress” may be output. The message output part 207 is implemented by the output device 24 of FIG. 1.

The signal transmitting/receiving part 208 sends and receives signals between the information processing apparatus 20 and the cleaning equipment 30, and between the information processing apparatus 20 and the multi-wavelength light source 11. Examples of the signal transmitted and received include, but are not limited to a control signal instructing the cleaning equipment 30 to start cleaning, a control signal for turning on and off the multi-wavelength light source 11, or changing the wavelength, and a report signal sent from the cleaning equipment 30 reporting the cleanup status or the cleaning result. The signal transmitting/receiving part 208 is implemented by the communication interface 25 of FIG. 1.

### Measurement and Judgment as to Necessity of Cleanup

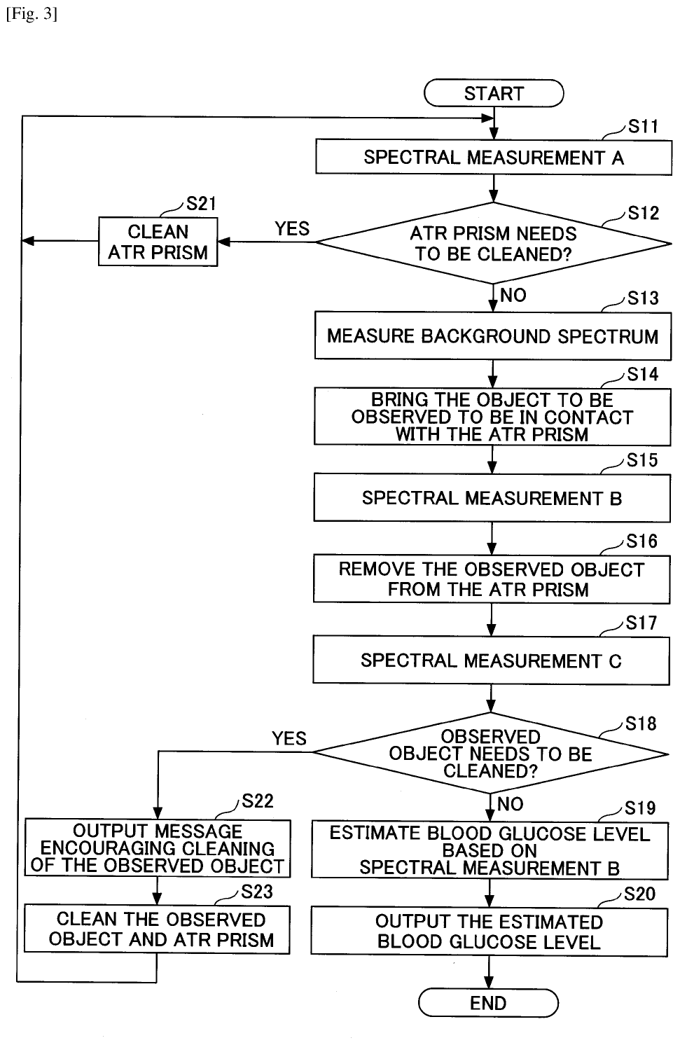

FIG. 3 is a flowchart of biological information measurement performed by the measuring system 100. The measuring flow includes determination as to necessity of cleanup. In this example, a blood glucose level is estimated as the biological information. First, a spectrum is acquired in a state where the ATR prism 12 is out of contact with the observed object (S11). This spectral measurement without physical contact between the ATR prism and the observed object is referred to as “spectral measurement A”. There is no particular restriction on the measurement condition, such as the number of scans or the resolution, for spectral measurement A, and appropriate conditions can be selected. The spectral measurement A may be carried out while the measuring device 10 is installed in the clean work station 300.

Based on the measurement result (including the absorbance) of the spectral measurement A, the information processing apparatus 20 determines whether the ATR prism 12 needs to be cleaned (S12). In this determination, a background spectrum acquired in the past may be used as the reference spectrum. The background spectrum used as the reference for judgement of necessity of cleanup of the ATR prism 12 is a spectrum acquired in advance from the ATR prism 12 without physical contact with an object to be observed, under the condition that the ATR prism 12 is judged as being clean. While the cleanness of the ATR prism 12 is confirmed, light is emitted from the multi-wavelength light source 11 and input to the ATR prism 12 to measure the background spectrum. The measured background spectrum may be stored in the memory 22, and it may be read out from the memory 22 when the determination of S12 is made.

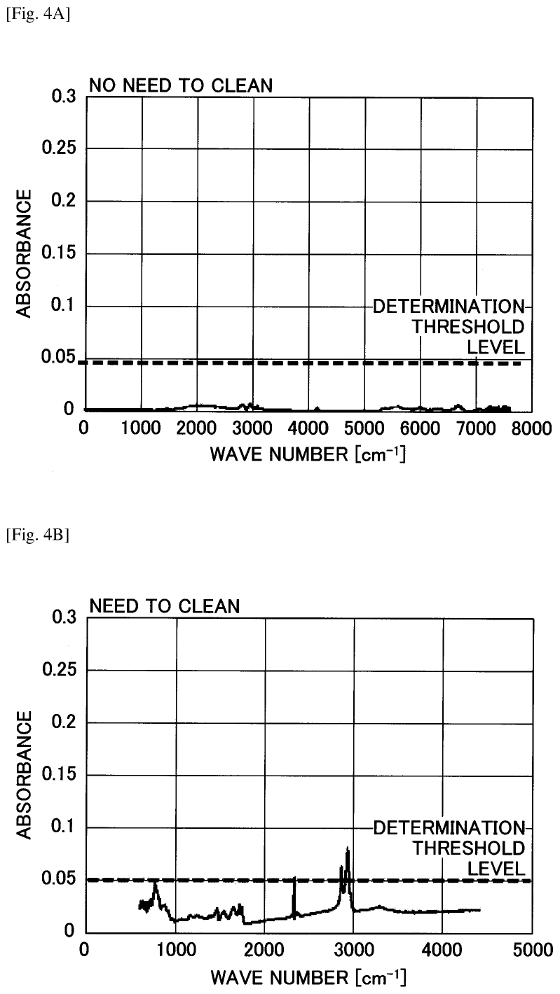

In the determination, if, for example, the difference between the spectrum of the spectral measurement A and the background spectrum is equal to or less than a predetermined level, it is determined that cleaning is unnecessary. If the difference exceeds the predetermined level, then it is determined that cleaning is necessary. If contamination due to the previous measurement remains on the ATR prism 12 even before the measurement of the blood glucose level, infrared light is absorbed by the contamination. In this case, the absorbance at the spectral measurement A becomes higher, and the difference from the background spectrum increases. Specific examples of judgement will be described later with reference to FIG. 4A and FIG. 4B.

If it is determined that the ATR prism 12 needs to be cleaned (YES in S12), a control signal instructing a cleanup of the ATR prism 12 is transmitted from the information processing apparatus 20 to the cleaning equipment 30, and a cleanup process is performed by the cleaning equipment 30 (S21). After the cleanup, the process returns to step S11, and the spectral measurement A is performed again using the clean ATR prism 12 without physical contact with the object to be observed.

The loops of steps S11, S12, and S21 are performed in order to remove the possibility of contamination of the ATR prism 12 due to residues from the previous measurements. By performing these steps, reliability of infrared spectroscopy for biological information measurement is improved.

If it is determined that cleanup of the ATR prism 12 is unnecessary (NO in S12), then a background spectrum is measured for actual blood glucose measurement (S13). The background spectrum for blood glucose measurement is acquired under the condition that the cleanness of the ATR prism 12 is ensured in S12. In FTIR spectroscopy, the absorption spectrum of the observed object is obtained from two measurements with a single beam, one with the presence of the observed object, and the other without the observed object. For this reason, the background spectrum is measured without physical contact between the ATR prism 12 and the observed object.

The measurement of the background spectrum in step S13 may be omitted, and the spectrum obtained from the spectral measurement A in step S11 may be used as the background spectrum for actual measurement of the blood glucose level, because the cleanness of the ATR prism 12 is ensured in the determination of step S12.

Next, the object to be observed is brought into contact with the ATR prism 12 (S14). In this example, the object to be observed is oral mucosa of the lips. Of course, the object to be observed is not limited to this example, and another body part, such as an eyelid or a fingertip, may be monitored. An absorption spectrum is measured in the state where the object to be observed is in contact with the ATR prism 12 (S15). This measurement of absorption spectrum is referred to as “spectral measurement B”. There are no restrictions on the measurement conditions for the spectral measurement B (such as the number of scans, the resolution, or the like). In this example, the spectral measurement B is performed under the same conditions as the spectral measurement A.

After the spectral measurement B is completed, the observed object is removed from the ATR prism 12 (S16), and the absorption spectrum is measured again without physical contact between the ATR prism 12 and the observed object (S17). This measurement of absorption spectrum performed after the measurement of blood glucose level is referred to as “spectral measurement C”.

When a living body is observed, some residue usually remains on the ATR prism 12 through the measurement. Therefore, after the blood glucose level has been measured, spectral measurement C is performed again without physical contact between the ATR prism 12 and the observed object. There are no restrictions on the measurement conditions for spectral measurement C (such as the number of scans, the resolution, or the like). In this example, the measurement conditions the same as those for spectral measurement A and spectral measurement B are employed.

If measurement is carried out on the lips, the ATR prism 12 is contaminated with food residues, saliva, or the like. If a fingertip or an eyelid is observed, the ATR prism 12 will be contaminated with sebum, sweat, or the like. Such contamination or residue will exist in the evanescent field during the spectral measurement B of S15, and the influence of the contamination is reflected in the spectrum obtained by spectral measurement C.

If a blood glucose level is measured using an unclean ATR prism 12 on which contamination or residue is left, or if the measurement is carried out on an unclean object to be observed, it is difficult to identify the features of the absorption spectrum derived from glucose, and as a result, the measurement reliability deteriorates. Meanwhile, the amount and the type of residue vary case by case. Even if the optical element or the object to be observed appears to be clean, the measurement result may still be affected, and vice versa. If cleanup of the observed part is required for every measurement, the burden on the subject or the user will increase. It is not easy for the subject or the user to judge whether the observed part is sufficiently clean from the appearance. Therefore, based on the result (including the absorbance) of the spectral measurement C, it is automatically determined whether or not the observed object needs to be cleaned (S18).

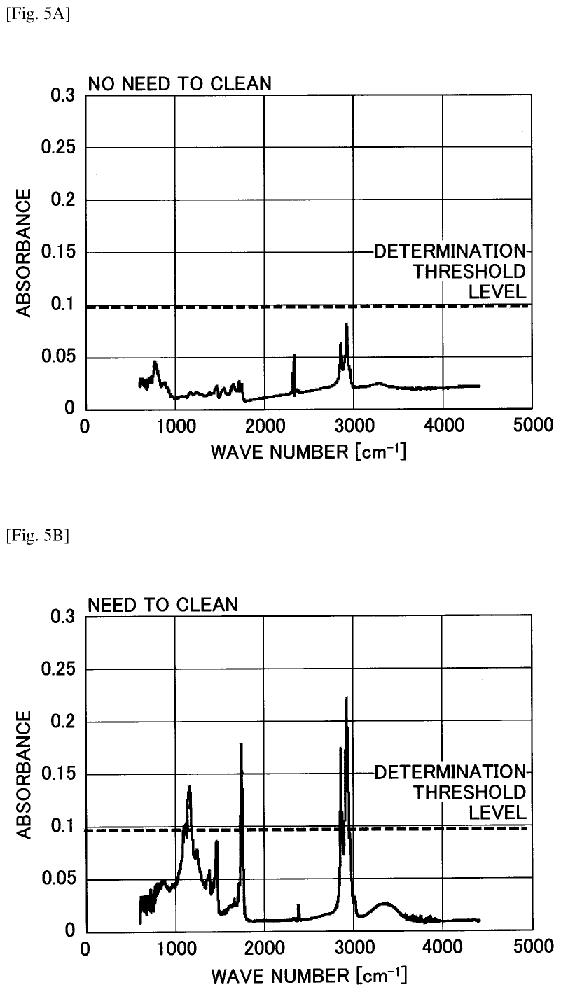

In S18, it is determined whether, for example, the difference between the spectrum obtained from the spectral measurement C and the background spectrum acquired in advance using a clean ATR prism 12 is equal to or less than a predetermined value. Specific examples of judgement will be described later with reference to FIG. 5A and FIG. 5B.

If it is determined that the observed object needs to be cleaned (YES in S18), the information processing apparatus 20 outputs a message encouraging the user to clean the observed part and remeasure (S22). A message such as “The observed part is not clean. Please measure again after cleanup” may be displayed on the display device; or such a voice message may be output. The message may either be text or images. The observed object is cleaned (S23). In this step, the ATR prism 12 may also be automatically cleaned by the cleaning equipment 30. After the observed object is cleaned, step S11 and the subsequent steps are repeated.

The flow from step S18 to S22, and S23 are performed to exclude undesirable measurements where the observed object is not clean and the absorption spectrum derived from glucose is adversely affected. By implementing these steps, measurement reliability of infrared spectroscopy is improved.

If it is determined that cleanup of the observed object is unnecessary (NO in S18), then the blood glucose level is estimated based on the absorption spectrum obtained in the spectral measurement B (S19). In the estimation, the background spectrum measured in S13 is used, together with the spectrum of the spectral measurement B, as has been described above. The estimated blood glucose level is output (S20), and the measurement process terminates.

According to the method of FIG. 3, biological information is measured after the cleanness of the ATR prism 12 and the observed object is ensured, and the accuracy and the reliability of measurement are improved.

FIG. 4A and FIG. 4B show how the necessity of cleanup of the ATR prism 12 is determined in S12 of FIG. 3. The horizontal axis represents the wavenumber, and the vertical axis represents the absorbance. The spectra shown in FIG. 4A and FIG. 4B are acquired from the ATR prism 12 which is not in contact with the object to be observed. Whether the ATR prism 12 needs to be cleaned may be determined based on the difference between the measured spectrum and the reference background spectrum acquired from the clean state, as has been described above. Alternatively, threshold determination may be performed, as shown in FIG. 4A and FIG. 4B, using the result of spectral measurement A acquired in step S11.

In FIG. 4A, there is almost no background noise, and the spectrum level is lower than the threshold level for determining the necessity of cleaning, over a predetermined range of wavenumber. In this case, no cleaning is required. On the other hand, in FIG. 4B, the amount of light absorption exceeds the threshold level at specific wavenumbers. If some residue has adhered to the surface of the ATR prism 12, the amount of light absorption exceeds the threshold level at a certain wavenumber, depending on the ingredient of the residue. In this case, it is determined that cleaning is required to remove the contamination from the ATR prism 12.

One reason why the ATR prism 12 needs to be cleaned prior to measurement is that the ATR prism 12 has been left uncleaned since the last measurement. In this case, the cell mucosa or sebum of the observed object (such as lips) tends to remain on the ATR prism 12. If such an uncleaned ATR prism 12 with contamination or residue remaining on the surface is used, absorption peaks are observed at the wavenumber of 2800 cm⁻¹ to 3000 cm⁻¹, as shown in FIG. 4B.

FIG. 5A and FIG. 5B show how the necessity of cleaning of the object to be observed is determined in S18 of FIG. 3. The spectra shown in these figures are acquired from spectral measurement C. In the background spectrum of the ATR prism 12 which has been in contact with the observed object and then removed from the observed object, absorption peaks appear in several wavenumber ranges.

If the object having been observed is clean to some extent, the absorbance is still small even if some absorption peaks appear, as shown in FIG. 5A. No matter how well the observed object is washed, a slight amount of cell mucosa or sebum adheres to the ATR prism 12 once the ATR prism 12 comes into contact with the observed object. Therefore, the threshold level for the determination of necessity of cleaning the observed object is set higher than the threshold level of FIG. 4A and FIG. 4B used for determination of the necessity of cleaning the ATR prism 12.

Even if an absorption peak is observed in the wavenumber range of 2800 cm⁻¹ to 3000 cm⁻¹, it is determined that there is no need to clean the observed object because the absorbance is below the threshold level, which means that the observed object is sufficiently clean.

When the observed object is not clean, the amount of residue adhering to the ATR prism 12 will increase, and absorption peaks exceeding the threshold level are observed as shown in FIG. 5B. With some food residue onto the ATR prism 12, peaks above the threshold level are observed at the wavenumber ranges 1700 cm⁻¹ to 1800 cm⁻¹, and 1000 cm⁻¹ to 1200 cm⁻¹, as well as in the wavenumber range 2800 cm⁻¹ to 3000 cm⁻¹.

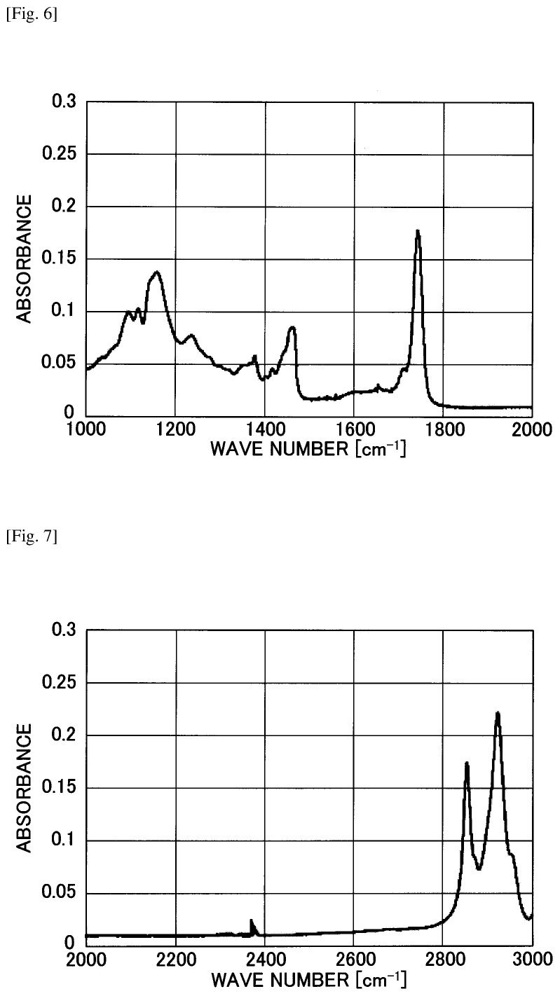

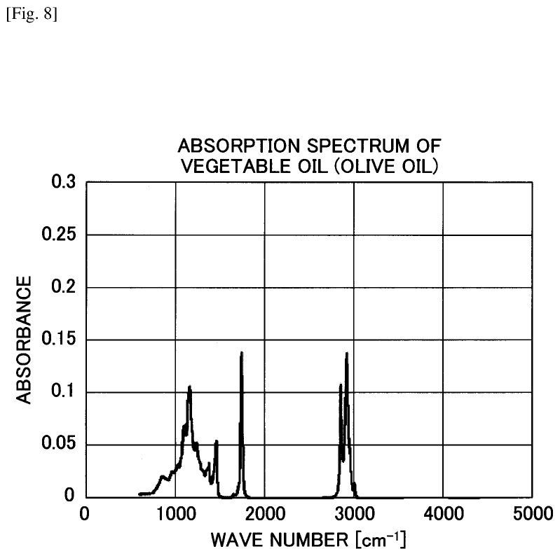

FIG. 6 is an enlarged view of the wavenumber range of 1000 cm⁻¹ to 2000 cm⁻¹ of FIG. 5B, and FIG. 7 is an enlarged view of the wavenumber range of 2000 cm⁻¹ to 3000 cm⁻¹ of FIG. 5B. FIG. 8 shows the absorption spectrum of vegetable oil just for information. Relying on FIG. 8, the peak at 1700 cm⁻¹ to 1800 cm⁻¹ in FIG. 6 is considered to be one derived from a double bond such as C=O, C=C, or C═N contained in a fat and oil component. The peaks in the range of 1000 cm⁻¹ to 1200 cm⁻¹ are considered to be derived from the chemical bonds of C—O, C—N, C—C, or the like contained in the fat and oil component. The peaks in the range of 2800 cm⁻¹ to 3000 cm⁻¹ in FIG. 7 are considered to be one derived from C—H bond contained in organic substances, such as cell mucosa, or oil and fat components.

By determining the necessity of cleaning the observed object based on the measurement result (including the absorbance) of the spectral measurement C in FIG. 3, undesirable measurement performed on an unclean object is excluded in advance, and the accuracy and reliability of biological information measurement are improved.

### Cleaning Process and Operation of Cleaning Equipment

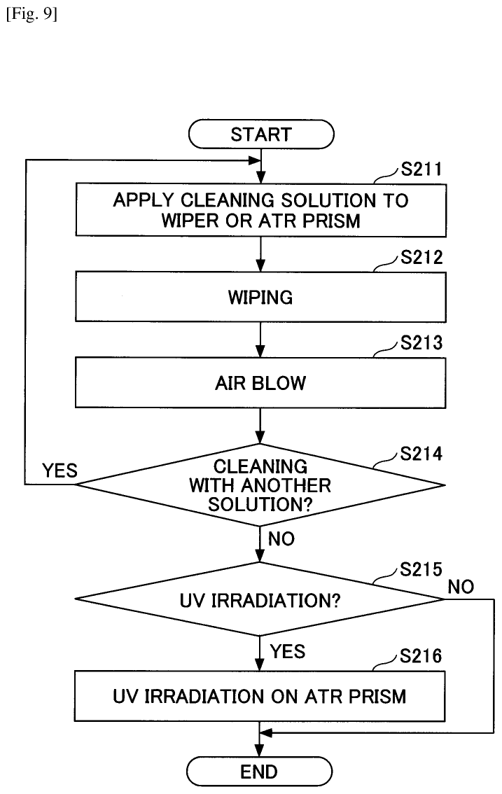

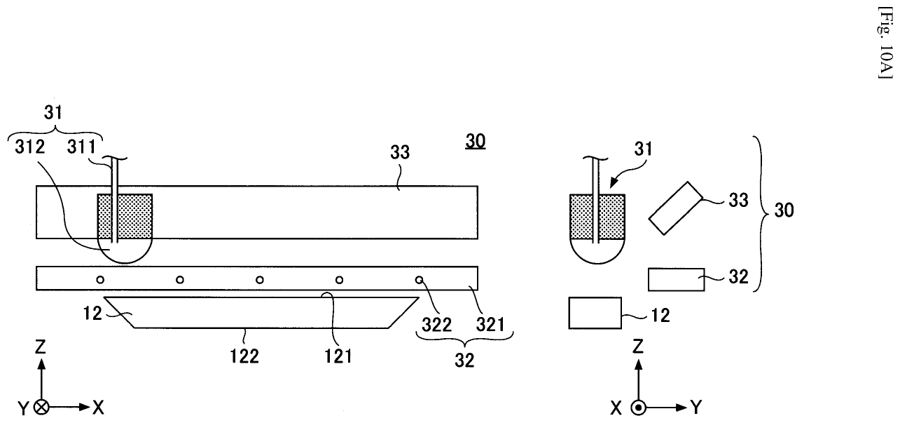

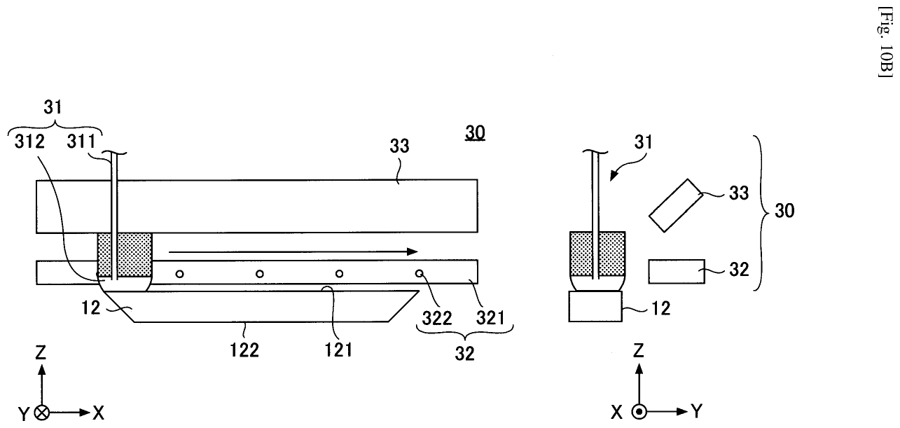

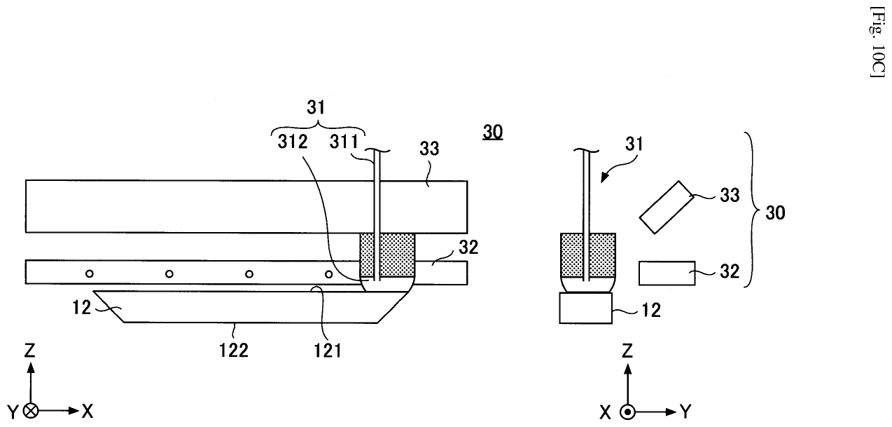

FIG. 9 is a flowchart of a cleaning process, and FIG. 10A to FIG. 10C illustrate examples of cleaning operations performed by the cleaning equipment 30. If it is determined in S12 of FIG. 3 that cleaning of the ATR prism 12 is necessary, the information processing apparatus 20 transmits a signal for cleanup instruction to the cleaning equipment 30. Upon receiving the signal, the cleaning equipment 30 starts the cleaning process.

The cleaning equipment 30 applies a cleaning solution to a wiper or the ATR prism 12 (S211) to wipe the surface of the ATR prism 12 (S212). As shown in FIG. 10A to FIG. 10C, the cleaning equipment 30 includes, for example, a wiping tool 31, an air blower 32, and an ultraviolet (UV) light source 33. The wiping tool 31 has a wiping member 312, which comes into contact with the surface of the ATR prism 12, and a solution nozzle 311 for supplying the cleaning solution to the wiping member 312. The direction in which the wiping member 312 moves during the wiping is in the X direction, the height direction of the cleaning equipment 30 is the Z direction, and the direction orthogonal to both the X direction and the Z direction is the Y direction.

The wiping member 312 and the solution nozzle 311 may be formed into a single unit. The cleaning solution may be supplied from a cleaning solution tank provided in the clean work station 300, and the solution flows through the solution nozzle 311 to the wiping member 312 provided at the nozzle tip. The cleaning solution is, for example, an alcohol disinfectant (ethanol solution). In place of the solution nozzle 311, a spray hole to spray the cleaning solution onto the surface of the ATR prism 12 may be used in the wiping tool 31.

The wiping tool 31 first stays at a non-contact position at which the wiping member 312 is not in contact with the ATR prism 12, as shown in FIG. 10A. Then, the wiping tool 31 moves in the −Z direction toward the wiping initial position, at which the wiping member 312 infiltrated by the cleaning solution comes into contact with the ATR prism 12, and then moves in the X direction, as indicated by the horizontal arrow in FIG. 10B, to wipe the surface of the ATR prism 12 with the wiping member 312. When the wiping member 31 has reached the wiping end position, as shown in FIG. 10C, the wiping process terminates.

The area on the ATR prism 12 to be wiped by the wiping tool 31 is a part or all of the totally reflecting surfaces 121 and 122, which potentially comes into contact with a living body. If the ATR prism 12 is held between the lips, the totally reflecting surfaces 121 and 122 of the ATR prism 12 are both wiped and cleaned.

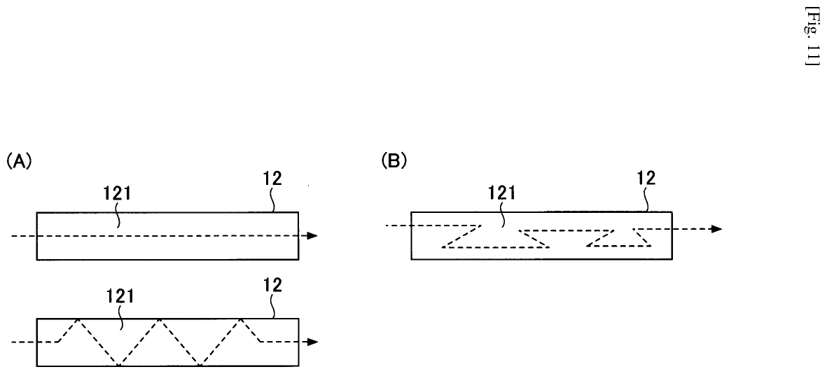

FIG. 11 shows a strategy for cleaning the ATR prism 12 in a top view of the totally reflecting surface 121. The locus of the center of the wiping member 312 extending on the totally reflecting surface 121 is indicated by a dashed arrow. As for the moving direction of the locus, it is desirable for the wiping member 312 to move only forward so as to prevent the wiped contamination from adhering again to the cleanup area.

Assuming that the position of the wiping member 312 is projected onto the long axis or the central axis of the ATR prism 12, it is desirable that the projected position shifts only in one direction. As shown in FIG. 11(A), the wiping member 312 may move linearly from the initial position at the left-hand side of the figure to the wiping end position at the right-hand side of the figure, or it may move in a zigzag manner. In either case, the wiping member 312 moves in a direction approaching the wiping end position. If the wiping member 312 moves backward as shown in FIG. 11(B), the wiping cleaning process may be inefficient.

It is desired for the wiping tool 31 not to pause the wiping member 312 on the ATR prism 12 during the cleaning process. Although, in the example of FIG. 11, wiping is performed from the left to the right of the figure, the wiping direction may be reversed. The number of wiping actions is not limited to one, and wiping may be repeated several times in the same direction.

Returning to FIG. 9, upon completion of the wiping, air blowing may be performed (S213). When the wiping tool 31 returns to the default position shown in FIG. 10A, the air blower 32 blows dry air onto the ATR prism 12 to dry the cleaning solution remaining on the surface of the ATR prism 12. The air blower 32 has a main body 321 and air holes 322. The air blower 32 may be configured so that the position of the main body 321 is movable, or that the direction of the air holes 322 is adjustable. By moving the main body 321 or changing the direction of the air holes 322, not only the vicinity of the surface of the ATR prism 12, but also the surrounding of the ATR prism 12 is dried. This configuration can prevent the IR spectrum from being affected by the volatile ethanol contained in the surrounding atmosphere.

When air blow is finished, it is determined whether to perform cleaning with another solution (S214). For example, if the information processing apparatus 20 determines that the peak appearing in the background spectrum acquired by the spectral measurement A is derived from fat or oil (YES in S214), wiping and air blow are repeated using another solution for removing the fat or oil from the surface of the ATR prism 12. In this case, the surface of the ATR prism 12 may be wiped off using a wiping tool different from the wiping tool 31 infiltrated with the ethanol disinfectant.

Whether to use a plurality of solutions in the wiping process may be instructed by a control signal when the information processing apparatus 20 drives the cleaning equipment 30. The cleaning equipment 30 may make determination in S214 according to the content of the control signal.

If recleaning with another solution is not required (NO in S214), then it may be determined whether to perform UV irradiation (S215). This determination may also be executed according to the content of the control signal from the information processing apparatus 20. If the information processing apparatus 20 determines that the peak appearing in the background spectrum acquired from the spectral measurement A is derived from bacteria or the like, a UV irradiation instruction may be included in the control signal for driving the cleaning equipment 30.

When UV irradiation is performed (YES in S215), the cleaning equipment 30 turns on the UV light source 33 and irradiates the ATR prism 12 with ultraviolet rays for a certain period of time (S216). The output surface of the UV light source 33 faces toward the surface of the ATR prism 12, and the surface of the ATR prism 12 is sterilized. Then, the cleaning process terminates.

It may be desirable that the cleaning process of FIG. 9 is automatically performed by the mechanism of the cleaning equipment 30 without manual operation of the operator; however, a part of the process, or a part of a certain step may be manually performed.

The cleaning process of FIG. 9 keeps the ATR prism 12 clean and improves the accuracy and reliability of infrared spectroscopy.

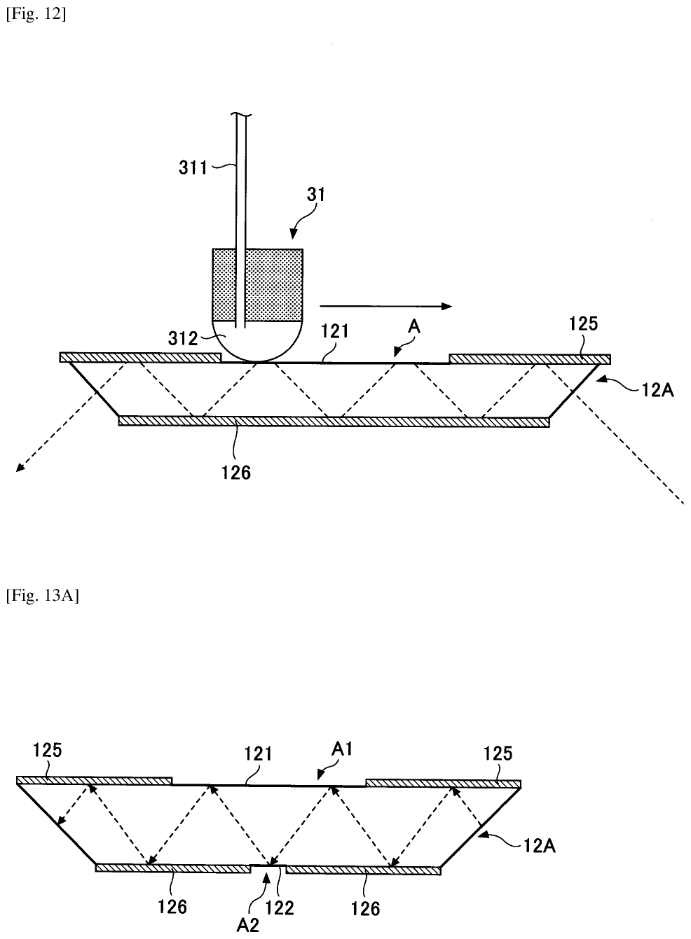

FIG. 12 shows a strategy of cleaning the ATR prism 12A. The ATR prism 12A has masks 125 and 126 on a part of the surfaces. The masks 125 and 126 are, for example, metal films formed by vapor deposition to define a monitoring sensitive region A. The monitoring sensitive region is a region that has a sensitivity with respect to the ATR measurement on the totally reflecting surface 121. More specifically, upon incidence of infrared light onto the ATR prism 12A, the evanescent field penetrates from the totally reflecting surface 121 into the living body, and the field attenuation effect by the living body occurs.

By providing the masks 125 and 126, the area that allows physical contact between the ATR prism 12A and the object to be observed is a constant area, and variations in the absorbance measurement can be reduced. In the wiping cleaning process for the ATR prism 12A, it is sufficient only to clean the area defined by the masks 125 and 126, that is, the exposed area of the totally reflecting surface 121 of the ATR prism 12A. It should be noted that, during the wiping process by the wiping tool 31, no infrared light is incident on the ATR prism 12A. By wiping only the monitoring sensitive region A in which the totally reflecting surface 121 is exposed, the amount of cleaning solution used for the cleaning process can be reduced, and time taken for the cleaning can be reduced. In addition, deterioration or peeling of the mask 125 or 126 can be prevented by not wiping the masks. Also, in recleaning using another solution, only the monitoring sensitive region A defined by the masks 125 and 126 is cleaned.

The totally reflecting surface 121 exposed in the monitoring sensitive region A is a surface that comes into physical or direct contact with a living body, and accordingly, the exposed surface may be cleaned repeatedly every time IR spectroscopy measurement is performed.

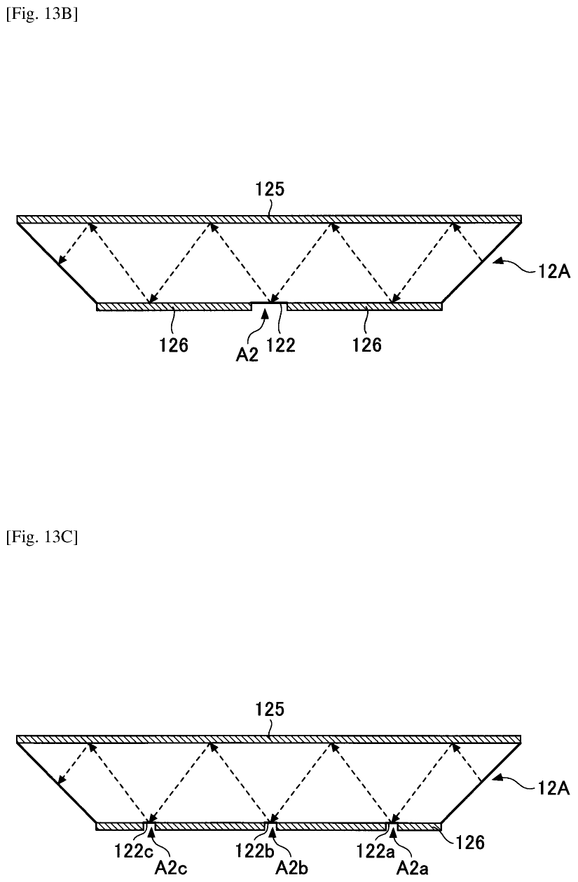

FIGS. 13A, 13B, and 13C show modifications of the ATR prism 12A. In FIG. 13A, a part of the totally reflecting surface 121 of the ATR prism 12A and a part of the totally reflecting surface 122 on the opposite side are exposed. The mask 125 defines a monitoring sensitive region A1 of the totally reflecting surface 121, and the mask 126 defines a monitoring sensitive region A2 of the totally reflecting surface 122.

The monitoring sensitive region A1 is located at or around the center of the totally reflecting surface 121, and the monitoring sensitive region A2 is located at or around the center of the totally reflecting surface 122. In the monitoring sensitive region A1, the light travelling through the ATR prism 12A is totally internally reflected multiple times (for example, twice) at the totally reflecting surface 121 as the interface with the living body, and the evanescent field penetrates into the living body. In the monitoring sensitive region A2, the light travelling through the ATR prism 12A is totally internally reflected once at the totally reflecting surface 122, and the evanescent field penetrates into the living body.

The mask configuration of FIG. 13A is advantageous especially when the ATR prism 12A is held between the lips so that the totally reflecting surfaces 121 and 122 both come into contact with the living body. With the ATR prism 12A held between the upper lip and the lower lip, a force tends to be generated at the central portion of the lips, so that the ATR prism 12A is stably kept in contact with the lips. On the other hand, it is not easy for the ends portion of the lips to apply force to hold an object between the lips. In addition, due to the individual differences in the size of the mouth, the contacting region between the living body and the ATR prism 12A tends to vary, and variation in measurement tends to increase. By defining the monitoring sensitive regions A1 and A2 with the masks 125 and 126, stable and reliable measurement is achieved.

In FIG. 13B, a part of the totally reflecting surface 122 of the ATR prism 12A is exposed, while the totally reflecting surface 121 is entirely covered with the mask 125. The mask 126 defines a monitoring sensitive region A2 at the center of the totally reflecting surface 122. When the ATR prism 12A is held between the lips, the lower lip is more likely to be pressed against the ATR prism under a stable pressing force. Therefore, the absorption spectrum is acquired mainly by making use of the field penetrating from the totally reflecting surface 122 into the lower lip.

In FIG. 13C, a part of the totally reflecting surface 122 of the ATR prism 12A is exposed at multiple areas, while the totally reflecting surface 121 is entirely covered with the mask 125. The mask 126 defines a plurality of monitoring sensitive regions A2a, A2b, and A2c on the totally reflecting surface 122. When the ATR prism 12A is held between the lips, the lower lip is more likely to be pressed against the ATR prism 12A under a stable pressing force. Therefore, the absorption spectrum is mainly acquired mainly by making use of the field penetrating from the totally reflecting surface 122 into the lower lip at multiple positions.

In general, the greater the number of total internal reflections, the greater the attenuation by the living body, and the measurement sensitivity increases. The measurement sensitivity of the configuration of FIG. 13C is higher that the configuration of FIG. 13B, because the number of total reflections that cause penetration of the field into the living body is increased. Thus, the blood glucose level can be measured with high sensitivity.

In any of the configurations of FIGS. 13A to 13C, stable measurement of biological information is achieved, regardless of individual differences, by defining a constant contacting area between the ATR prism and a living body. For the measurement, necessity of cleaning the ATR prism 12A is automatically determined, and the ATR prism 12A is automatically cleaned as necessary. Therefore, the totally reflecting surface(s) of the ATR prism 12A is kept clean in any configuration of FIG. 13A to FIG. 13C.

Although the present invention has been described above based on examples of specific configurations, the measurement method and system configuration of the present invention are not limited to the above-described process and configuration. The measurement target is not limited to a blood glucose level, and the embodiments can be applied to the measurement of other biological information such as protein, or blood tumor DNA. As described above, a plurality of light sources that output light at different wavelength ranges may be combined, in place of the multi-wavelength light source 11.

According to the process and configuration of the embodiment, the optical element used for the measurement and an object to be observed are kept clean throughout the measurement, and a biometric information measurement technique with improved reliability is achieved.

This International Patent Application is based upon and claims priority to earlier Japanese Patent Application No. 2021-036544, filed on Mar. 8, 2021, the entirety of which is incorporated herein by reference.

1. A method of measuring biological information using an optical element with a totally reflecting surface, which is to be brought into contact with an object to be observed, comprising:
after the optical element has been in contact with the object to be observed, acquiring an absorbance at the totally reflecting surface in a state where the optical element is not in contact with the object to be observed; and
determining by an information processing apparatus whether the object to be observed needs to be cleaned, based on the absorbance.

2. The method as claimed in claim 1, further comprising:
if it is determined by the information processing apparatus that the object to be observed needs to be cleaned, outputting a signal encouraging cleaning of the object to be observed.

3. The method as claimed in claim 1, further comprising:
if it is determined by the information processing apparatus that the object to be observed needs to be cleaned, cleaning the object to be observed; and
after the object to be observed is cleaned, bringing the optical element to be in contact with the object to be observed and acquiring an absorbance of the object to be observed.

4. A method of measuring biological information using an optical element with a totally reflecting surface, which is to be brought into contact with an object to be observed, comprising:
before the optical element comes into contact with the object to be observed, acquiring an absorbance at the totally reflecting surface at a first wavenumber; and
determining by an information processing apparatus whether or not the optical element needs to be cleaned, based on the absorbance at the first wavenumber, the first wavenumber being in a range of 1700 cm⁻¹ to 1800 cm⁻¹ or 2800 cm⁻¹ to 3000 cm⁻¹.

5. The method as claimed in claim 4, further comprising:
if it is determined by the information processing apparatus that the optical element needs to be cleaned, automatically cleaning the optical element; and
after automatic cleaning, bringing the object to be observed to be in contact with the optical element and acquiring the biological information.

6. The method as claimed in claim 5, wherein:
the automatic cleaning includes pressing a wiping member onto the totally reflecting surface of the optical element and moving the wiping member in a predetermined direction so that a position of the wiping member projected onto a center axis of the totally reflecting surface changes only in one direction.

7. The method as claimed in claim 5, wherein:
the automatic cleaning includes air blowing the optical element.

8. The method as claimed in claim 5, wherein:
the automatic cleaning includes ultra-violet irradiation to the optical element.

9. (canceled)

10. A measuring system comprising:
a measuring device having an optical element with a totally reflecting surface, a light source, and a photodetector configured to detect a light output from the light source, reflected by the totally reflecting surface, and output from the optical element; and
an information processing apparatus configured to process a detection result of the photodetector,
wherein the light source is configured to output light beams of different wavenumbers including a first wavenumber, the first wavenumber being in a range of 1700 cm⁻¹ to 1800 cm⁻¹ or 2800 cm⁻¹ to 3000 cm⁻¹, and
wherein the information processing apparatus acquires an absorbance measured at the totally reflecting surface at the first wavenumber in a state where the optical element is not in contact with an object to be observed.

11. The measuring system as claimed in claim 10, wherein:
the information processing apparatus determines whether the optical element needs to be cleaned, based on the absorbance at the first wavenumber.
